(19) United States
(12) Patent Application Publication
(10) Pub. No.: US 2020/0171282 A1
(43) Pub. Date: Jun. 4, 2020

(54) SYSTEM AND METHOD FOR TREATMENT OF THE CLAUDIFICATION OF THE SUPERFICIAL FEMORAL AND PROXIMAL POPLITEAL ARTERY

(71) Applicant: Micro Medical Solutions, Inc., Wilmington, MA (US)

(72) Inventor: Gregory Sullivan, Wilmington, MA (US)

(21) Appl. No.: 16/702,767

(22) Filed: Dec. 4, 2019

Related U.S. Application Data
(60) Provisional application No. 62/775,055, filed on Dec. 4, 2018.

(51) Int. Cl. A61M 25/10

(52) U.S. Cl. CPC A61M 25/104; A61M 2025/1043

(57) ABSTRACT

A system and accompanying methods for treating a blockage of the superior femoral artery including utilization of a puncture insertion mechanism, an introducer sheath, a guide catheter and an expandable device. Entry into the body is established through the popliteal and advancing retrograde through the popliteal artery toward the lower superior femoral artery region and in some embodiments utilizing an introducer sheath, a guide catheter and a balloon are featured.

### CROSS REFERENCE TO RELATED APPLICATION

This application takes the benefit of and claims priority to U.S. Provisional Patent Application Ser. No. 62/775,055 filed on Dec. 4, 2018, the contents of which are herein incorporated by reference.

### BACKGROUND OF THE INVENTION

### Field of the Invention

The instant system relates generally to medical devices, including surgical and medical delivery systems, and more particularly to a method for the treatment of the claudification of the SFA and PPA.

### Description of the Related Art

Peripheral arterial disease (PAD) is a common manifestation of atherosclerosis affecting 5 million adults in the United States, with an age-adjusted prevalence of 4% to 15% and increasing up to 30% with age and the presence of cardiovascular risk factors. In this article we focus on lower extremity PAD and specifically on the superficial femoral and proximal popliteal artery (SFPA), which are the most common anatomic locations of lower extremity atherosclerosis.

Chronic lower extremity ischemia, also classified as PAD, is a common condition managed by vascular specialists. The primary etiology is atherosclerosis. Atherosclerotic stenosis or occlusion of the peripheral arterial tree results in arterial insufficiency and end-organ (limb) ischemia. PAD is a major contributor to morbidity, reduced quality of life (QOL), and mortality in an increasing elderly demographic in the Western world.

As the population ages, it is anticipated that the prevalence of peripheral vascular disease will increase. Within the past decade there has been an unprecedented evolution of the endovascular technologies and vital improvements are expected in the next decade. Percutaneous procedures will continue to replace open surgery. The chief challenge in the management of peripheral arterial disease would be retooling of the health system to focus on identifying patients with PAD and taking the enormous opportunity and responsibility to refine and aggressively manage the atherosclerotic risk factors in these patients.

The superficial femoral artery is a continuation of the common femoral artery at the point where the profunda femoris branches. It is the main artery of the lower limb and is, therefore, critical in the supply of oxygenated blood to the leg. Over the past decade there has been a remarkable advancement in the endovascular treatment of lower extremity PAD with the introduction of new interventional techniques and devices, and specifically on the SFPA, which are the most common anatomic locations of lower extremity atherosclerosis.

Currently, endovascular treatment of SFPA disease is indicated for individuals with significant disability due to intermittent claudication or critical limb ischemia when clinical features suggest a reasonable likelihood of symptomatic improvement with endovascular intervention, there has been an inadequate response to exercise or pharmacological therapy and when there is a favorable risk-benefit ratio. Claudication is defined as muscular pain, cramping, aching, or discomfort in the lower limb, reproducibly elicited by exercise and relieved within 10 minutes of cessation.

Endovascular surgery is an innovative, less invasive procedure used to treat problems affecting the blood vessels, such as an aneurysm, which is a swelling or “ballooning” of the blood vessel. The surgery involves making a small incision near each hip to access the blood vessels. An endovascular graft, which is a special fabric tube device framed with stainless steel self-expanding stents, is inserted through the arteries in a catheter, a long, narrow flexible tube, and positioned inside the aorta. Once in place, the graft expands and seals off the aneurysm, preventing blood from flowing into the aneurysm. The graft remains in the aorta permanently.

Apart from the clinical and angiographic criteria for selection of patients for endovascular treatment, for stenoses of 50-75% diameter by angiography, intravascular translesional pressure gradients have been recommended to determine whether these lesions are hemodynamically significant and to predict patient improvement after revascularization. Although there is no consensus on the diagnostic translesional pressure gradient criteria, the most widely accepted criteria utilize a mean gradient of 10 mmHg before or after vasodilators; or a mean gradient of 5 mmHg and peak systolic gradient of 10, 15 or 20 mmHg; or 15% peak systolic pressure gradient after administration of a vasodilator. The TASC II consensus emphasizes more anatomic criteria and recommends endovascular revascularization for type A lesions and surgery for type D lesions, whereas endovascular treatment is preferred for type B lesions and surgery for good-risk patients with type C lesions.

For some time, surgical revascularization has been the main form of treatment. Surgical treatment of lower extremity ischemia is indicated for patients with claudication and significant functional disability or critical limb ischemia, after failure of conservative or endovascular therapy, who have a reasonable likelihood of symptomatic improvement, favorable limb arterial anatomy and low cardiovascular risk for surgical revascularization. With the evolution of endovascular technology and interventional techniques, as well as their equivalent efficacy, lower cost and lower peri-procedural risk, surgery has become a second-line revascularization option and is currently recommended only for TASC D lesions.

Furthermore, as surgery should be avoided in patients younger than 50 years old, since they have a more virulent form of atherosclerosis and subsequently a higher frequency of graft failure requiring revisions and replacement, minimally invasive surgery is plainly the better choice. Minimally invasive surgery is a surgery minimizing surgical incisions to reduce trauma to the body. This type of surgery is usually performed using thin-needles and an endoscope to visually guide the surgery. During minimally invasive surgery, doctors use a variety of techniques to operate with less damage to the body than with open surgery. In general, minimally invasive surgery is associated with less pain, a shorter hospital stay and fewer complications.

Normally, once the decision to proceed with surgical intervention is undertaken, the type of revascularization should be elected based on different variables, such as location and severity of disease, anatomy, general medical condition, prior revascularization attempts and the desired outcome. As a general rule, in patients with combined inflow and outflow disease, inflow problems are corrected first, since improvement of the inflow may diminish the symptoms of claudication and reduce the likelihood of distal graft thrombosis from low flow. In the case of superficial femoral and proximal popliteal artery (SFPA) disease, two major factors that can modify the result of the procedure are the type of the conduit and the site of the distal anastomosis.

The superior rates of immediate and long-term patency rates favor autogenous vein grafts as opposed to prosthetic conduits for both above- or below-the-knee bypasses. In some studies, the 5-year patency rates of femoropopliteal bypass grafts are reported as 80% for vein grafts, 75% for above-the-knee synthetic grafts and 65% for below-the-knee synthetic grafts. Patients undergoing surgical bypass for lower extremity ischemia should be entered into a clinical surveillance program that consists of interval history and vascular exam as well as measurement of resting and, if possible, post-exercise ABIs and duplex imaging of the entire length of the graft with measurements of peak systolic velocities and calculation of the velocity ratios across all lesions in the immediate postoperative period and at regular intervals (usually every 6 months) for at least 2 years.

Major amputation in patients with critical or acute limb ischemia should be reserved only when the limb is unsalvageable, i.e., when there is overwhelming infection that threatens the patient's life, extensive necrosis or refractory ischemic rest pain.

Currently, a wide variety of established and evolving endovascular techniques to treat PAD including percutaneous transluminal angioplasty (PTA) with balloon dilation, stents, endografts, atherectomy, laser, cutting balloons, drug-coated balloon angioplasty, cryoplasty, percutaneous thrombectomy and brachytherapy exist.

The challenge for the vascular specialist is to determine whether the nature and severity of presenting symptoms correlate with the degree of chronic arterial insufficiency present or whether alternative etiologies, such as neuropathy, inflammation, infection, lymphatic or venous disease, and repetitive trauma, are more likely responsible. Definitive diagnosis is derived from detailed historic and physical examination findings correlated with appropriately directed noninvasive vascular laboratory and adjunctive imaging studies.

Patients with PAD may present with a spectrum of symptoms ranging in severity from none to varying degrees of claudication to severe or “critical” limb ischemia. CLI has been traditionally defined as (1) persistent, recurring ischemic rest pain requiring opiate analgesia for more than 2 weeks and (2) ankle systolic pressure less than 50 mmHg or toe systolic pressure less than 30 mmHg (or absent pedal pulse in patients with diabetes). Ischemic rest pain typically is nocturnal, worsens with elevation, and is relieved by dependency. Pedal pulses are absent; dependent rubor, elevation pallor, and calf muscle atrophy are frequent accompaniments. CLI also includes ischemic foot ulceration and gangrene in the setting of ankle systolic pressure less than 50 to 70 mmHg or toe systolic pressure less than 40 mmHg in patients without diabetes (<50 mmHg in diabetics).

All patients with PAD require comprehensive medical management and risk factor modification. Revascularization (either open bypass or endovascular intervention) is indicated in patients who remain symptomatic and significantly limited despite adequate risk factor modification, exercise, and medical management. The primary goal of intervention, in patients with lifestyle-limiting claudication, is to improve exercise tolerance and hence QOL. Patients with rest pain, tissue loss, and gangrene are at greater risk for limb loss and cardiovascular mortality (stroke, myocardial infarction) associated with systemic atherosclerosis than those who present with claudication alone. Revascularization in the critical limb ischemia (CLI) cohort is focused on wound healing and functional limb salvage as well as symptomatic relief and improvement in QOL.

The vascular specialist must first determine, given the underlying disease burden, the severity of ischemic and infectious complications as well as the patient's comorbidities, functional status, and anticipated longevity. Once it is decided that revascularization will improve the patient's functional status and QOL, these same variables, in concert with anatomic assessment of the location, extent, and severity of occlusive arterial lesions will determine whether endovascular, open, or hybrid revascularization options are indicated. When bypass is selected as the preferred revascularization option, the goals of preoperative planning involve delineation of diseased arterial segment(s), identification of the most appropriate arterial inflow source, selection of the optimal bypass target for maximal outflow and target bed perfusion, and selection of the best available conduit. In practice, conduit availability is almost always a critical, rate-limiting factor because good quality, autogenous vein conduit is preferred in almost every circumstance.

Adequate preoperative planning depends on a thorough history and detailed physical examination and thus the delineation of the relevant arterial anatomy on the index limb is facilitated by high quality, noninvasive vascular laboratory studies (ankle-brachial index and toe pressure measurements). These are supplemented by arterial color duplex ultrasound imaging. Arterial duplex is extremely accurate in the assessment of iliofemoral and femoropopliteal arterial occlusive disease but less so for infrageniculate (tibial-peroneal) lesions. Duplex enables differentiation of stenosis from occlusion and determination of lesion length and degree of calcification. Cross-sectional imaging studies such as computed tomography angiography (CTA) or magnetic resonance arteriography (MRA) may add complementary information, but most experienced operators prefer the precision and resolution inherent in catheter-based, intraarterial contrast arteriography for definitive preoperative planning, especially when bypass will be required to distal calf or pedal targets.

PAD is a coronary artery disease equivalent. Therefore, preoperative risk evaluation for overall cardiovascular-related mortality represents a component of preoperative planning. In most patients with stable or minimally symptomatic coronary disease, preoperative risk-reduction efforts are best focused on optimizing medical management. Frequently, this includes statin and antiplatelet therapy, β-blockade, and optimization of hypertension management. The surgical plan should be tailored to each patient's needs based on extent of disease, conduit availability, and realistic long-term functional potential. Infrainguinal bypass may originate from the common, superficial, or deep femoral artery or the popliteal artery with a bypass target of the popliteal, tibial, or pedal/plantar arteries. The positioning, choice of incisions, and surgical techniques are dictated by type of bypass procedure deemed most appropriate under the circumstance.

Discussing some state of the art techniques, including techniques utilizing vessel exposure, the patient may be placed in a supine position and a Foley catheter is inserted. Arms may be tucked to facilitate intraoperative prebypass and completion angiography.

Regarding placement of an incision, the common femoral artery (CFA) is located on a line between the pubic tubercle and anterior iliac spine, two fingerbreadths lateral to pubic tubercle. Palpation. of the inguinal ligament and femoral pulse or direct arterial visualization with duplex imaging can localize the CFA bifurcation and guide optimal incision placement. Even when pulseless due to excessive calcification or occlusive disease, the CFA may be localized by reliance on anatomic landmarks and direct palpation, recognized as a firm tubular structure positioned within the femoral sheath.

The vertical groin incision is most commonly employed to provide optimal access to the entire length of the CFA. This should be created coaxially along the artery itself, continued from the inguinal ligament distally, and aimed at the medial aspect of the knee. The incision can be extended superiorly or inferiorly to increase arterial exposure as necessary to achieve optimal inflow.

Alternatively, especially in obese patients with substantial abdominal pannus, a curvilinear incision can be placed 1 cm below and parallel to the inguinal ligament to avoid potential skin maceration and wound complications that may accompany vertical incisions in this situation. Although the proximal superficial femoral and deep femoral arteries can be exposed via this incision, such a curvilinear or oblique incision limits further distal arterial exposure. It therefore would not be selected if an extensive common and deep femoral artery endarterectomy is anticipated as potentially necessary to optimize inflow. The incision is carried sharply through the subcutaneous tissue and superficial fascia.

Investigating the state of the art for dissection and control of the common, superficial, and proximal deep femoral arteries, deep to the subcutaneous tissue and superficial fascia, the dissection is extended longitudinally, even when using an oblique incision, to optimize the length of femoral exposure. Depending on the depth of dissection and subcutaneous adiposity, self-retaining Weitlaner or cerebellar retractors are carefully placed to optimize exposure while avoiding traction injury to femoral nerve branches or the common femoral vein. Further dissection through the femoral sheath exposes the anterior surface of the femoral artery.

The dissection plane should remain centered directly over the femoral artery. Encountering venous structures indicates medial deviation from the optimal plane; exposure of the iliopsoas muscle, femoral nerve fibers, or lymphatic vessels is an indication of lateral deviation. An increasing incidence of femoral incisional complications, including wound edge necrosis and separation, lymphatic leaks, femoral neuropraxia, and venous injuries are associated with incorrectly placed inguinal incisions for femoral exposure.

Dissect directly along the CFA both proximally and distally. Placement of silastic vessel loops around the femoral artery and its larger branches aids in retraction, dissection, and mobilization. Proximal dissection is continued along the CFA to the inguinal ligament. The inguinal ligament may be divided to aid in exposure or to enable extended endarterectomy. Caution is necessary in this area, as a prominent femoral vein tributary crosses anteriorly over the CFA in this area and is prone to injury if not identified, ligated, and divided early in the dissection. Inadvertent injury to this “vein of pain” produces retraction and troublesome bleeding.

The medial and lateral femoral circumflex arteries, important collaterals in iliofemoral arterial occlusive disease, are identified at level of the inguinal ligament and individually controlled with removable clips or silastic vessel loops. Use of the former reduces clutter in the wound during endarterectomy or creation of the proximal anastomosis.

As the dissection proceeds distally, an abrupt change in caliber marks the femoral bifurcation and the origins of the deep (also known as “profunda femoris” in Latin) and superficial femoral arteries (SFA). The latter continues distally in the same plane; the former usually courses posteriorly and laterally away from the femoral bifurcation. After silastic loops are placed on each vessel, gentle upward traction on the CFA or SFA may help bring the deep femoral artery into view. The lateral circumflex iliac vein may course anteriorly over the origin of the deep femoral artery and should be ligated and divided to optimize exposure and control of the first segment of this vessel.

Medial and distal dissection provides extended exposure of the proximal SFA. This vessel only occasionally has small branches in its proximal segment. A sensory branch of the femoral nerve may be present crossing the SFA from lateral to medial. Transection may result in medial thigh discomfort. Even extended femoral bifurcation dissections rarely require division of femoral nerve branches, which should be avoided to minimize postoperative paresthesias and dysesthesias.

Exposure of the distal portions of the deep femoral artery often enables use of shorter vein conduit in distal leg bypass or may improve outflow from proximal revascularization procedures (iliac angioplasty and stenting or aortofemoral bypass). These segments are easily exposed from either posteromedial or anteromedial approaches. The approach should be dictated by the indication (inflow sources or outflow target); an additional consideration is the necessity to obtain exposure in a native field, either in the setting of prior dissection or femoral graft infection.

Incisions are placed along either the medial (anteromedial approach; or lateral borders (posterolateral approach) of the sartorius muscle The dissection plane is developed through the subcutaneous tissue and fascia, passing lateral or medial to the sartorius, respectively. Mobilize and retract sartorius muscle laterally or medially, depending on approach.

When the artery in the groin, the femoral artery, is stenosed or blocked, an operation can be used to remove the plaque from the artery in order to improve flow and circulation. The artery is close to the surface here under the skin, and therefore the surgical procedure is not too invasive. An alternative treatment would be angioplasty with a stent if required. This can be done with access to the arterial system from the opposite groin or arm to perform this procedure.

Traditionally the long term results of surgery to remove the plaque when it is localized to the femoral artery have been better than angioplasty. However in certain patient's not suitable for surgery, and with improving; technology, angioplasty is being used more frequently Both treatments can therefore be used, the vascular surgical team will decide which treatment is the better approach for the situation at hand.

The surgical procedure can be performed under general or local anaesthetic. The plaque is removed from the artery. A patch is often used to repair the artery and avoid narrowing. The patient will normally stay in hospital 1 or 2 nights following this procedure. If there is also disease with narrowed or blocked arteries elsewhere above the groin or further down the leg, the endarterectomy operation (removal of material on the inside of the artery) can be combined with angioplasty and stenting to treat these other areas at the same time. This combined or “hybrid” procedure is more common now and it achieves a better restoration of the circulation than the endarterectomy procedure alone.

Further investigating the current state of lower extremity revascularization for patients with claudication and critical limb ischemia. Regarding treatment of the SFA for claudicative diseases, currently revascularization through an endovascular approach has become the default method for therapy in most, if not all, vascular beds (intricate network of minute blood vessels that ramifies through the tissues of the body or of one of its parts). The treatment for peripheral arterial obstructive disease in the lower extremity is no exception.

The quandary faced by the industry, and thus many of the questions asked revolve around whether or not to treat only when the claudication is at its worse or treat more aggressively and early, as this impacts potential heart-healthy lifestyles. Other key issues broach whether drug-delivery balloons and the current stent designs are not the correct systems for the SFA and whether changes to stent technologies—woven stents, alternatively designed stents or very conformable stents—define the future treatment directives.

Thus, PAD of the SFA is the most common cause of intermittent claudication. Atherosclerotic disease of the SFA is localized to the region of Hunter's canal. An isolated occlusion or stenosis of the SFA often results in decreased perfusion of the leg, resulting in demand related, reversible, ischemic pain localized to the calf. Ischemic rest pain and tissue loss, also known as CLI, are uncommon manifestations of isolated SFA disease. CLI is more commonly observed when occlusive disease of the SFA is combined with occlusive disease involving the below knee popliteal artery or tibial arteries.

As discussed above, percutaneous transluminal angioplasty (PTA) is a minimally invasive technique for treatment of superficial femoropopliteal artery (SFPLA) obstructions or occlusions in patients with intermittent claudication as well as critical limb ischemia. With the introduction of endovascular stents, the problems of elastic recoil and residual stenoses due to arterial dissection could be resolved and initial reports of stenting for the treatment of occlusive atherosclerotic disease of the SFPLA have exhibit positive results. However, subsequent studies demonstrated that exaggerated neo-intimal hyperplasia (proliferation and migration of vascular smooth muscle cells primarily in the tunica intima, resulting in the thickening of arterial walls and decreased arterial lumen space) in the stented segment frequently leads to instent restenosis.

Also, cutting balloons may be suited for as the balloon-mounted microtomes guarantee smooth lumen gain within the stent, without the risk of vessel wall perforation. Initial reports of the use of the cutting balloon for the treatment of occlusive atherosclerotic disease of the SFPLA show promising results, indicating that the problems of elastic recoil and residual stenoses due to arterial dissection might be resolved. The cutting balloon has four tiny microtomes (<0.1 mm height) on the outside, which cut the fibrous plaque during expansion of the balloon. Consequently the problem of elastic recoil is ideally addressed, additionally less trauma is exercised on the vessel wall during dilatation of the balloon.

This might be achieved by a reduction of vessel wall trauma, vessel wall inflammation and consequently reduced neointimal formation. Although the indications for cutting balloon angioplasty (CB-PTA) in the SFA includes significant residual stenosis or in-stent restenosis, there are currently no published randomized controlled trials (RCT) comparing PTA vs. CB-PTA for any specific condition. This lack of data led us to initiate a RCT comparing primary PTA vs. CB-PTA for treatment of in-stent restenoses in patients with intermittent claudication or critical limb ischemia with TASC category A-B in the femoropopliteal artery.

Historically, endovascular treatment of the SFA was first described by Charles Dotter wherein he used Teflon coated dilators to sequentially angioplasty the SFA in an 82-year-old woman to treat critical limb ischemia that was considered non-operable. Subsequently, Gruntzig popularized the concept of catheter directed balloon angioplasty. Angioplasty disrupts the atherosclerotic plaque by displacing it radially and results in stretching of the adventitia thereby increasing the lumen diameter in the treated vessel. By definition, a dissection is created and if significant, can be flow limiting.

Currently, the most commonly utilized endovascular revascularization options are percutaneous transluminal angioplasty (PTA) with provisional stenting or primary stenting. Provisional or selective stenting is indicated for the treatment of flow limiting dissections and/or persistent, hemodynamically significant stenoses or recoil after PTA. This approach is recommended by the Tran-Atlantic inter-Society Consensus document II (TASC II) when treating SFA disease. However, both PTA alone and primary stenting can successfully treat SFA disease. Therefore, the debate continues as to which endovascular treatment is superior.

PTA has the advantage of being inexpensive and technically simpler than primary stenting, and is especially well looked upon as only PTA alone avoids utilization of foreign bodies that may be a potential stimulus for intimal hyperplasia. Another benefit of using PTA alone includes avoiding material fatigue and fractures associated with stenting wherein fatigue and fracture may result from the torque and deformation of the femoropopliteal arteries that occurs during flexion of the knee joint.

Further, the advent of lower profile angioplasty balloons allows PTA of the SFA through vascular sheaths as small as 4 French. Smaller sheath diameters result in fewer complications, and therefore, are considered safer. In addition, the ease of re-intervention or bypass of an angioplastied arterial segment following PTA may be advantageous. The presence of a stent may impede endovascular re-intervention if a re-stenosis results in occlusion of the stented arterial segment. Moreover, angioplasty preserves collateral vessels that may be compromised by stent placement.

The clear need for advancement is illustrated through the need to treat chronic limb ischemia arise when it results in the limb threatening conditions of rest pain and tissue loss and possible loss of limb illustrate the exigency. Debilitating symptoms of intermittent claudication, a condition in which cramping pain in the leg is induced by exercise, typically caused by obstruction of the arteries, may occur and intermittent claudication is the most common manifestation of isolated SFA arterial disease. Though SFA disease is often present in patients with CLI, it is frequently seen in conjunction with multi-level arterial occlusive disease.

Remote superficial femoral artery endarterectomy (RSFAE) is a procedure to remove plaques inside the SFA. The femoral artery is the main blood vessel in your thigh that carries blood and oxygen to the legs. Plaques are fat, cholesterol, or tissues that are clogged in the inner wall of the artery. When plaques build up inside the superficial femoral artery, blood flow to the legs may be decreased. RSFAE may be done to relieve problems caused by a narrowed or blocked artery. Problems that may happen include severe pain in the hip, thigh, calf, or foot, and trouble when walking. Having these problems may decrease a person's ability to do his daily activities and affect his quality of life.

With RSFAE, the plaque that blocks the artery is removed through a small incision (cut) in the groin. The groin is the area where your abdomen (stomach) meets your upper leg. Caregivers strip, cut, and remove the plaque by using different tools inserted through the SFA. This is done using a special type of x-ray as a guide. RSFAE may be followed by other procedures, such as angioplasty and stenting to open the artery using a small, high pressure balloon and implant a metal or plastic stent, in the area where the blockage was removed, to keep the artery open.

In an advantageous manner, utilizing a retrograde approach to treat SFA with a PTA approach augments the overall capabilities. As an alternative, a guide catheter can be advanced in stages with a balloon or a guidewire can be advanced independently to access the most proximal location in the vessel prior to introduction of the balloon catheter and paving can advance proximal to distal.

### SUMMARY OF THE INVENTION

The instant system, method and set of accompanying apparatuses allow for retrograde access to the superficial femoral artery for treatment of blockages and restoration of proper blood flow to vital pathways.

In one embodiment, it is an object of the instant system to introduce a retrograde stepwise paving approach to treat the SFA and particularly the lower SFA.

It is an additional object of the instant system to introduce a method wherein a vessel more superficial compared to femoral is utilized for access and thus requiring a smaller puncture. In one embodiment, an objective of the instant system to introduce a method wherein a vessel more superficial compared to femoral is utilized for access and thus requiring a smaller puncture which is particularly advantageous in the more obese patients.

An object of the instant system to introduce a series of devices and a method for endovascular treatment of SFPA disease is indicated for individuals with significant disability due to intermittent claudication or critical limb ischemia.

In one embodiment, it is an object of the instant system to introduce a series of devices and a method for direct access not dealing with branch vessels allowing for navigation without wire. The column strength of guide catheter can break through heavily calcified lesion and operate in combination with low profile balloon catheter can pave way proximal through the SFA.

In one embodiment, it is an object of the instant system to introduce a series of devices and a method for advancing distal to proximal in order to reduce the onset of shower of emboli below the knee into the territory of the foot. Positioning an embolic protection device at or near the puncture site will permit safe removal of dislodged plaque or emboli generated during the access and angioplasty procedure.

In one embodiment, it is an object of the instant system to introduce a series of shorter devices and an accompanying method which permit significantly greater user control while treating claudication or critical limb ischemia within the lower extremities, peripheral arterial disease and specifically on the superficial femoral and proximal popliteal artery (SFPA), lower SFA

In one embodiment, it is an object of the instant system to utilize a wide variety of established and evolving endovascular techniques to treat PAD including percutaneous transluminal angioplasty (PTA) with balloon dilation, stents, endografts, atherectomy, laser, cutting balloons, drug-coated balloon angioplasty, cryoplasty, percutaneous thrombectomy and brachytherapy exist.

As an alternative, the guide catheter can be advanced in stages with the balloon or the guidewire can be advanced independently to access the most proximal location in the vessel prior to introduction of the balloon catheter and paving can advance proximal to distal (i.e in the direction of the puncture site).

The foregoing has outlined the more pertinent and important features of the present invention in order that the detailed description of the invention that follows may be better understood, and the present contributions to the art may be more fully appreciated. It is of course not possible to describe every conceivable combination of components and/or methodologies, but one of ordinary skill in the art may recognize that many further combinations or permutations are possible. Accordingly, the novel architecture described below is intended to embrace all such alterations, modifications, and variations that fall within the spirit and scope of the appended claims.

There has thus been outlined, rather broadly, the more important features of the retrograde approach to treatment in the area of the SFA, including all applicable methods, systems or series of accompanying systems, apparatuses and embodiments in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty, which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

To the accomplishment of the foregoing and related ends, certain illustrative aspects are described herein in connection with the following description and the annexed drawings. These aspects are indicative of the various ways in which the principles disclosed herein can be practice and all aspects and equivalents thereof are intended to be within the scope of the claimed subject matter. Other advantages and novel features will become apparent from the following detailed description when considered in conjunction with the drawings.

### BRIEF DESCRIPTION OF THE DRAWINGS

Advantages of the present system will be apparent from the following detailed description of exemplary embodiments thereof, which description should be considered in conjunction with the accompanying drawings, in which having thus described the system in general terms, reference will now be made to the accompanying drawings, which are not necessarily drawn to scale, and wherein:

FIG. 1 illustrates cross sectional view of a human limb further illustrating the anatomy of the limb with the arteries highlighted and further illustrating an access point for a retrograde approach to treating blockages of the lower portion of the superficial femoral artery or lower SFA.

FIG. 2 illustrates a cross sectional view of the procedural tract illustrating the access point for retrograde approach to treating blockages of the lower SFA. Further illustrated is the puncture site for sheath access through Popliteal behind the knee. Herein, the system being utilized introduces a 3F introducer sheath, a guide catheter for advancing toward an area of dense plaque within the lower SFA and a balloon in a deflated orientation. The blood flow direction is illustrated and thus retrograde access is being performed herein.

FIG. 3 illustrates cross sectional view of the access point for retrograde approach to treating blockages of the lower SFA. Further illustrated is the puncture site for sheath access through Popliteal behind the knee. Herein, the system being utilized introduces a 3F introducer sheath, a guide catheter for advancing toward an area of dense plaque within the lower SFA and a balloon in an inflated orientation in order to free up the blockage in a retrograde paving technique which opens up the calcified lesion.

FIG. 4. illustrates a right side view, detailed cutaway of the anatomy of limb showing access point for retrograde approach to treating lower SFA, illustrating the full path for entry and delivery from the popliteal entry to superficial femoral artery.

FIG. 5. illustrates a left side view, detailed cutaway of the anatomy of the limb showing access point for retrograde approach to treating lower SFA, illustrating the entirety of the entry and delivery path from the popliteal entry to superficial femoral artery in the color green. In this figure, the pathway is illuminated from the puncture site through popliteal behind the knee to the blockage are in the lower SFA.

### DETAILED DESCRIPTION OF THE SEVERAL EMBODIMENTS

The detailed description set forth below in connection with the appended drawings is intended as a description of presently preferred embodiments of the system and does not represent the only forms in which the present system may be constructed and/or utilized. The description sets forth the functions and the sequence of steps for constructing and operating the system in connection with the illustrated embodiments.

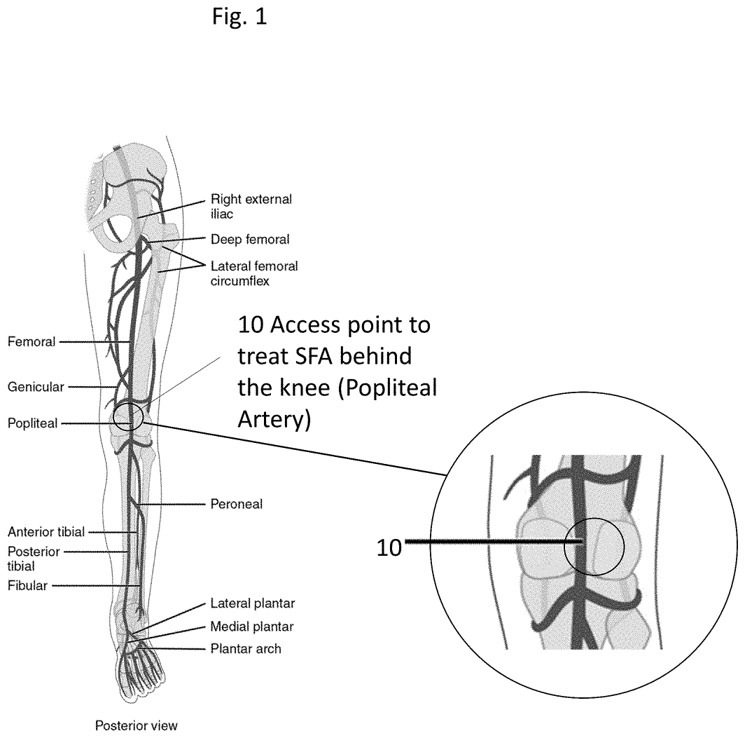

FIG. 1 illustrates a cross sectional view of a human limb further illustrating the anatomy of the limb with the arteries highlighted and further illustrating an access point 10 for a retrograde approach to treating blockages of the lower superficial femoral artery (SFA).

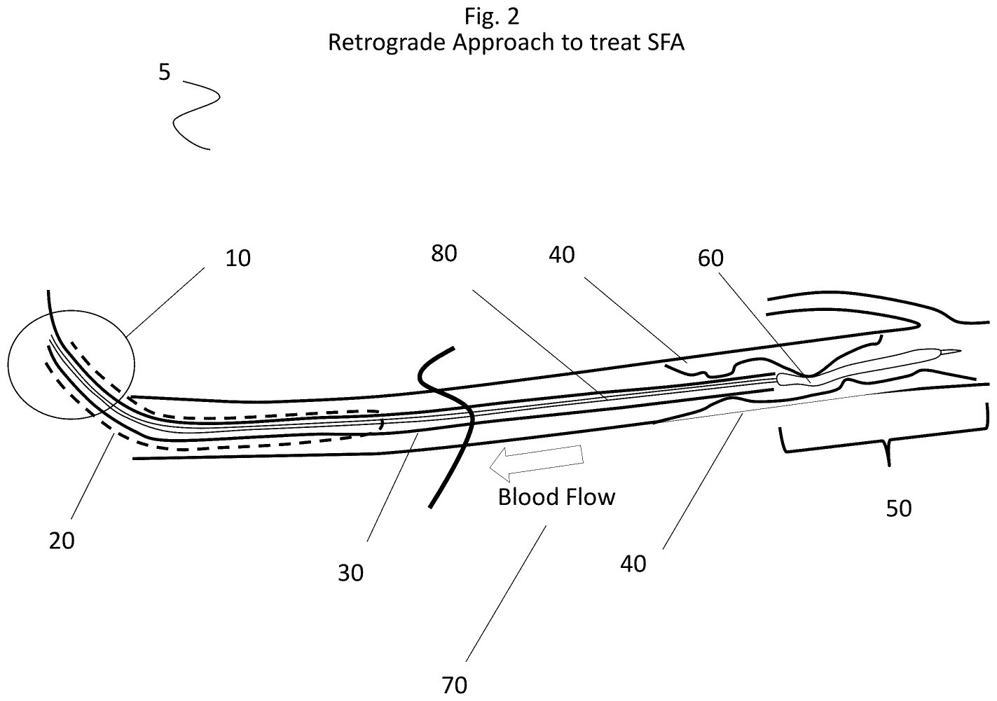

FIG. 2 illustrates a cross sectional view of the procedural tract 5 illustrating the access point 10 for retrograde approach to treating blockages of the lower SFA, further illustrating the instrumentation utilized at the access point 10 or the puncture site and for sheath access through the Popliteal and popliteal artery 15 behind the knee. Herein, the system being utilized introduces an introducer sheath 20, a guide catheter 30 for advancing toward an area of dense plaque 40 within the lower SFA 50 and a balloon 60 in a deflated orientation.

In one embodiment, the introducer sheath 20 utilized may be a three French or 3F introducer sheath 20. The blood flow direction indicator 70 is illustrated, thus denoting that retrograde access is being performed herein. Again, a guide wire 80 may also be utilized in conjunction with the currently described system.

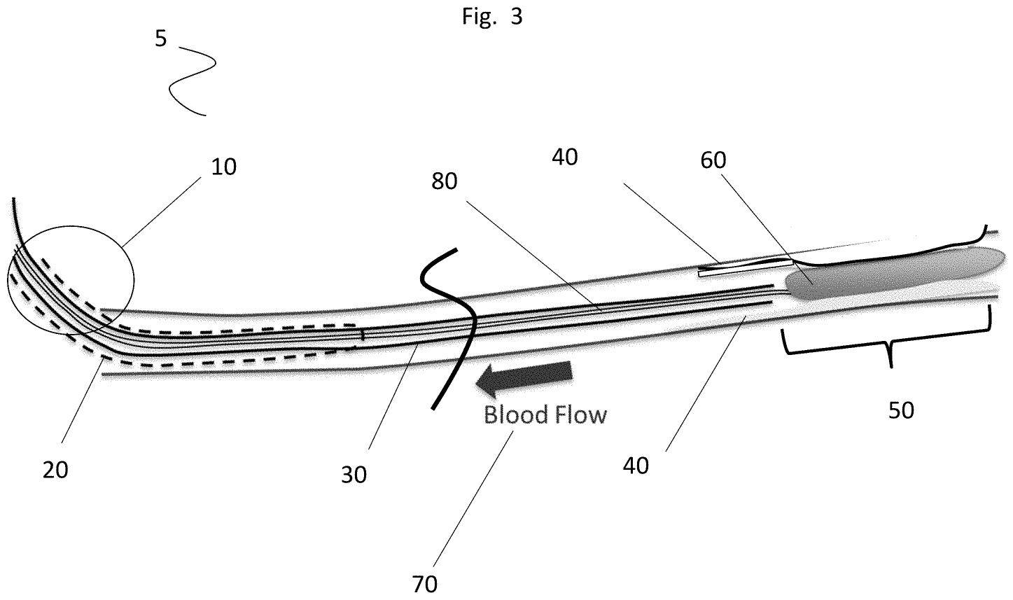

FIG. 3 illustrates a further cross-sectional view of the procedural tract 5 illustrating the access point 10 and the system deployed further illustrating the access point 10 through the Popliteal and popliteal artery 15 behind the knee for retrograde approach to treating blockages of the lower SFA 50. Further illustrated is the puncture site for sheath access through Popliteal behind the knee. Herein, the system being utilized introduces a 3F introducer sheath 20, a guide catheter 30 for advancing toward an area of dense plaque 40 within the lower SFA 50 and the balloon 60, now in the inflated orientation in order to free up the blockage in a retrograde paving technique which opens up the calcified lesion.

Once again, the blood flow direction indicator 70 is illustrated, thus denoting that retrograde access is being performed herein. Additionally illustrated herein is a guidewire 80, which may also be utilized.

Further, as the direction of the blood flow is opposed to direction of operation of the system and thus the initial the paving as illustrated constitutes retrograde access being performed. In an alternative methodology, the guide catheter may be advanced in stages with the balloon.

In an additional embodiment, the guidewire may be advanced independently to access the most proximal location in the vessel prior to introduction of the balloon catheter and paving can advance proximal to distal (i.e in the direction of the puncture site).

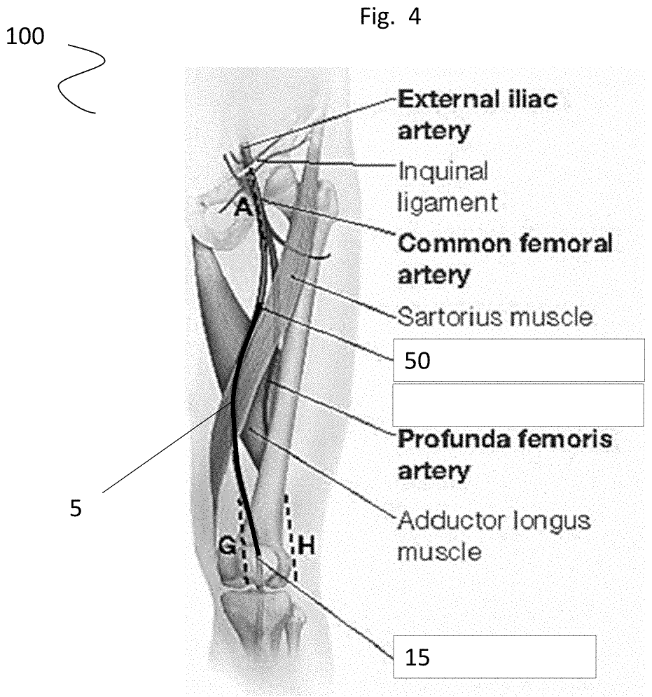

FIG. 4. illustrates a right-side view of a more detailed cutaway of the anatomy of the limb 100 illustrating the entirety of the entry and delivery tract 5 from the popliteal artery 15 entry to superficial femoral artery in the color green. In this figure, the pathway is illuminated from the puncture site through popliteal behind the knee to the blockage are in the lower SFA.

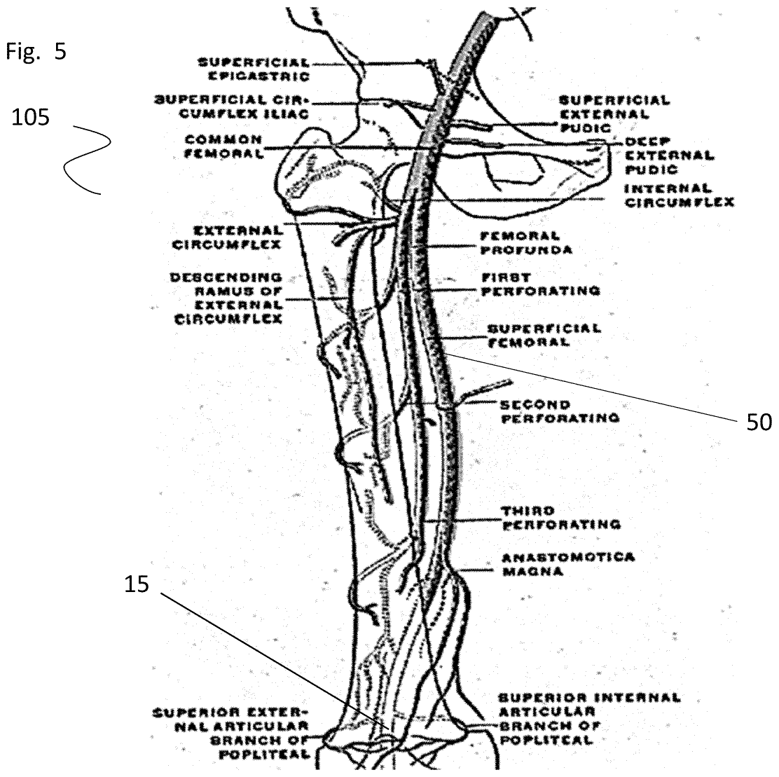

FIG. 5. illustrates a left side view, detailed cutaway of the anatomy of the limb 105 showing access point for retrograde approach to treating lower SFA, illustrating the entirety of the entry and delivery path 5 from the popliteal artery 15 entry to superficial femoral artery in the color green. In this figure, the pathway is illuminated from the puncture site through popliteal behind the knee to the blockage are in the lower SFA.

In one embodiment, a method of treating a blockage of the superior femoral artery (“SFA”) is disclosed, which involves the steps of:

selecting an area behind a knee of the affected limb;

creating a puncture site behind the knee;

entering the puncture site through the popliteal;

advancing retrograde through the popliteal toward a lower SFA region;

utilizing an introducer sheath having a proximal end, a distal end, an opening at the distal end, and a passage in communication with the opening at the distal end, the one or more prostheses being movably disposed in the passage;

utilizing a guide catheter;

utilizing an expandable member;

positioning the distal end of the introducer sheath and the expandable member at the treatment site; and

proximally retracting the introducer sheath relative to the expandable member and thereby cause the one or more prostheses to pass through the opening of the introducer sheath for expansion at the treatment site via the expandable member.

Additionally, the method includes the use of a guide wire and where it is advanced in stages. In another embodiment, the introducer sheath comprises a three (3) French introducer sheath.

In yet another embodiment, the method includes additional steps of advancing the guidewire independently to access the most proximal location in a vessel and introducing a balloon. In another embodiment, the method involves the steps of advancing the guide catheter and paving proximal to distal in the direction of the puncture site and advancing distal to proximal in order to reduce a shower of emboli below the knee into the territory of the foot.

In an alternate embodiment, the method of treating a blockage of the superior femoral artery comprises the step of positioning an embolic protection device in the region proximal to the puncture site to permit safe removal of dislodged plaque or emboli generated during the access and angioplasty procedure and comprises the step of removing dislodged plaque or emboli generated during the access and angioplasty procedure.

Another aspect of the method of treating a blockage of the superior femoral artery of claim 1 includes the step of advancing directly from the popliteal to the SFA and access not dealing with branch vessels allowing for navigation without wire and optionally utilizing a system which does not include the guide wire for navigation of the popliteal and SFA.

Furthermore, the method of treating a blockage of the superior femoral artery of further comprises the step of selecting or calibrating a measure of column strength of the guide catheter can break through heavily calcified lesion and the step of operating in combination with a low-profile balloon catheter to advance proximally through the SFA.

In yet another embodiment, the method of treating a blockage of the superior femoral artery of further comprises the step of advancing the guide catheter in a method selected from the group consisting of in stages with a balloon and independently, and may also include the steps of:

advancing the guide catheter independently;

accessing the most proximal location in the vessel prior to introduction of the balloon catheter; and

paving proximally to distally in the direction of the puncture site to reduce shower of emboli below the knee into the territory of the foot.

Finally, in yet another embodiment, the method of treating a blockage of the superior femoral artery comprises the step of positioning an embolic protection device at or near the puncture site will permit safe removal of dislodged plaque or emboli generated during an access and angioplasty procedure and the step of utilizing a retrograde paving technique to open the calcified lesion.

In yet another embodiment, a system for treating a blockage of the superior femoral artery is disclosed, which includes:

a puncture mechanism;

selecting an area behind a knee of the affected limb;

creating a puncture site behind the knee;

entering the puncture site through the popliteal;

advancing retrograde through the popliteal toward a lower SFA region;

an introducer sheath;

a guide catheter; and

a balloon.

Additionally, the system for treating a blockage of the superior femoral artery may utilize a low profile balloon catheter to pave proximal through the SFA, while maximizing the column strength of the guide catheter to break through heavily calcified lesion.

In yet another embodiment, a method of delivering one or more prostheses to a treatment site is disclosed, involving the steps of providing a catheter, and using a sheath having a proximal end, a distal end, an opening at the distal end, and a passage in communication with the opening, the one or more prostheses being movably disposed in the passage with an expandable member near the distal end of the sheath, the one or more prostheses being positionable over the expandable member, and a valve member near the distal end of the sheath adapted for selectively retaining a first set of prostheses within the passage and to engage the one or more prostheses whereby the one or more prostheses may be moved relative to the expandable member by moving the sheath; positioning the distal end of the sheath and the expandable member at the treatment site; and proximally retracting the sheath relative to the expandable member to move the valve member and thereby cause a first set of prostheses to pass through the opening of the sheath for expansion at the treatment site via the expandable member while retaining a second set of prostheses within the passage.

1. A method of treating a blockage of the superior femoral artery (“SFA”) comprising the steps of:
selecting an area behind a knee of the affected limb;
creating a puncture site behind the knee;
entering the puncture site through the popliteal;
advancing retrograde through the popliteal toward a lower SFA region;
utilizing an introducer sheath having a proximal end, a distal end, an opening at the distal end, and a passage in communication with the opening at the distal end, the one or more prostheses being movably disposed in the passage;
utilizing a guide catheter;
utilizing an expandable member
positioning the distal end of the introducer sheath and the expandable member at the treatment site; and
proximally retracting the introducer sheath relative to the expandable member and thereby cause the one or more prostheses to pass through the opening of the introducer sheath for expansion at the treatment site via the expandable member.

2. The method of treating a blockage of the superior femoral artery of claim 1 further comprising utilizing a guide wire.

3. The method of treating a blockage of the superior femoral artery of claim 1 wherein the introducer sheath comprises a three (3) French introducer sheath.

4. The method of treating a blockage of the superior femoral artery of claim 1 wherein the guide catheter is advanced in stages.

5. The method of treating a blockage of the superior femoral artery of claim 1 further comprising the step of advancing the guidewire independently to access the most proximal location in a vessel.

6. The method of treating a blockage of the superior femoral artery of claim 1 further comprising the step of introducing a balloon.

7. The method of treating a blockage of the superior femoral artery of claim 4 further comprising the step of advancing the guide catheter and paving proximal to distal in the direction of the puncture site.

8. The method of treating a blockage of the superior femoral artery of claim 1 further comprising the step of advancing distal to proximal in order to reduce a shower of emboli below the knee into the territory of the foot.

9. The method of treating a blockage of the superior femoral artery of claim 1 further comprising the step of positioning an embolic protection device in the region proximal to the puncture site to permit safe removal of dislodged plaque or emboli generated during the access and angioplasty procedure.

10. The method of treating a blockage of the superior femoral artery of claim 1 further comprising the step of removing dislodged plaque or emboli generated during the access and angioplasty procedure.

11. The method of treating a blockage of the superior femoral artery of claim 1 further comprising the step of advancing directly from the popliteal to the SFA and access not dealing with branch vessels allowing for navigation without wire.

12. The method of treating a blockage of the superior femoral artery of claim 1 further comprising the step of utilizing a system which does not include the guide wire for navigation of the popliteal and SFA.

13. The method of treating a blockage of the superior femoral artery of claim 1 further comprising the step of selecting or calibrating a measure of column strength of the guide catheter can break through heavily calcified lesion.

14. The method of treating a blockage of the superior femoral artery of claim 1 further comprising the step of operating in combination with a low-profile balloon catheter to advance proximally through the SFA.

15. The method of treating a blockage of the superior femoral artery of claim 1 further comprising the step of advancing the guide catheter in a method selected from the group consisting of in stages with a balloon and independently.

16. The method of treating a blockage of the superior femoral artery of claim 1 further comprising the steps of:
advancing the guide catheter independently;
accessing the most proximal location in the vessel prior to introduction of the balloon catheter; and
paving proximally to distally in the direction of the puncture site to reduce shower of emboli below the knee into the territory of the foot.

17. The method of treating a blockage of the superior femoral artery of claim 1 further comprising the step of positioning an embolic protection device at or near the puncture site will permit safe removal of dislodged plaque or emboli generated during an access and angioplasty procedure.

18. The method of treating a blockage of the superior femoral artery of claim 1 further comprising the step of utilizing a retrograde paving technique to open the calcified lesion.
